SEQUENCE LISTING (18 sequences)

SEQ ID NO: 1 - DNA, 1575 bp, Drosophila melanogaster
  CDS (1)..(1572)
atggagtggtcgctcatcatatcgattctgctcaactgcctctacgccagcgcctccgcgctcgacgaggattgccccaccttggacgatggggacagtttgtcgcaaacacagctgctggatcgcttaacccatggctgccgctacgatcgtttggaacgacccataacttattcggaaactggccagcggctaccggtggacgtctatatgcgcgcctacatctacttcatgcagaacctggaagcacacgatctacagttcaagatctatgcgctcctccaaatgcgatacctcgatccgcgtctcaactttcgcaacgtaagtcccaagcgcaggcagccgattttgggcgaggagcacctgagaaactcgctctggatgccccacatatttctagccaatgagcgcgactccagtattttgggtctcacggagaaggacatcctcacctcgatatcgcccgatggcacagtgatcgtttcgaatcgcatcaaggcgacactctattgctggctcaaccttaagaagttcccattcgatgagcagcactgctcgacggtgctggagagttggatgtacaatacgtcggaactggtcctccactgggagcagaagcgtccgataacctacgatggggcactgcatctcaccgagtttgtgctgcagcgttcctggtcaaacgagacggtaatcaatgccgatttgagtgacctgcgtcatggagcctttgccggcaactacagctccctcagcttcaccgttcacctgacgcgagtggttggcttttacctgatggactactttctaccctccatgctgattgtggccatatcctgggtatcgttctggctgcaagcagatcaggcgccgccgcgcatcacgctgggcaccagcaccctgctgacattcattaccctggcctcggcacagggaaagactctgccaaaggttagctacatcaaggtgtcagaggtgtggttcctgggctgcaccatcttcatatttggcagcatggtggagttcgcctttgttaacacgatttggcgtcgtaagaagagtgtgcccgtcaagaaactgaacagcaaacacatcctgaagtcaactctatcgccgaatctgctcagaagacgcagtcacgccagatccagatccttttcgggcacggctctgcataagacagcggataccggctcgctgggcggtgccggtttcaacaattacctaaccgtcaatttgcccatcaccacgattaaggagggccaagttctggatcagcagaggaccagccgcgtcgtccagaagatggatgttaactttgtggagagtgcctttggatccatgggctctgcccagtccactggagcgatcagtgctgccagcagcagccaaactttgaccaacaataaccgaacggagagcgcggggcaccaggaggagatcgatctgagtggctggaccacgctaacgccccaggaaatagccatgtggattgatagtcgagccagattcgtttttccgctctcctttctcgtctttaatctgtttttctggacatttgtctactgtatttaa

SEQ ID NO: 2 - protein, 524 aa, Drosophila melanogaster
MEWSLIISILLNCLYASASALDEDCPTLDDGDSLSQTQLLDRLTHGCRYDRLERPITYSETGQRLPVDVYMRAYIYFMQNLEAHDLQFKIYALLQMRYLDPRLNFRNVSPKRRQPILGEEHLRNSLWMPHIFLANERDSSILGLTEKDILTSISPDGTVIVSNRIKATLYCWLNLKKFPFDEQHCSTVLESWMYNTSELVLHWEQKRPITYDGALHLTEFVLQRSWSNETVINADLSDLRHGAFAGNYSSLSFTVHLTRVVGFYLMDYFLPSMLIVAISWVSFWLQADQAPPRITLGTSTLLTFITLASAQGKTLPKVSYIKVSEVWFLGCTIFIFGSMVEFAFVNTIWRRKKSVPVKKLNSKHILKSTLSPNLLRRRSHARSRSFSGTALHKTADTGSLGGAGFNNYLTVNLPITTIKEGQVLDQQRTSRVVQKMDVNFVESAFGSMGSAQSTGAISAASSSQTLTNNNRTESAGHQEEIDLSGWTTLTPQEIAMWIDSRARFVFPLSFLVFNLFFWTFVYCI

SEQ ID NO: 3 - DNA, 1269 bp, Drosophila melanogaster
  CDS (1)..(1266)
atgcaaagcccaactagcaaattggtagaattcaggtgcctgattgcgttggcaatatatttgcacgcgctggagcaatcgatccagcactgccattgtgttcatggttacagaaataacacggagagcgccgagctggtctcccactacgagtcgagtctttcgctcccggacattttgcccattccctcaaagacgtacgacaagaaccgggctcccaagctcctcggccagcccacagtagtctacttccatgtcacggtcctctccctggactccattaacgaggagtctatgacctatgtgacggacatcttccttgcacaaagctggcgtgatcctcgcctgcggttgcctgagaacatgagtgagcagtatcgcatattggatgtcgactggttgcacagcatttggcggcccgattgcttctttaagaacgccaaaaaggtcaccttccatgagatgagcattcccaatcactatctctggctgtaccacgacaaaacgctgctctatatgtccaaactcacgttggtcctgtcgtgcgccatgaagtttgagtcctatccgcatgacacgcaaatctgctccatgatgatcgagagtttatcccatacggtggaagatttggttttcatttggaacatgaccgacccacttgtggttaacacggagattgagttgccgcagctagacatatcaaataactacacaaccgactgtactatagagtactcaacaggtaacttcacctgcctggccattgtgttcaacctgcgccgacgcctgggttaccatttgttccacacctacatcccctcggctctgattgtggtcatgtcttggatatcgttttggataaaaccagaagcgatacccgcccgtgtaactctgggagtgacctcactgctaaccctggccacccagaatacccagtcgcaacaatcgctgccgccggtttcgtatgtcaaggctatagacgtctggatgtcgtcctgttcggtgtttgtattcctttctctgatggaatttgcagtggtcaacaattttatgggaccggtggccacaaaggcaatgaaggggtattcggacgagaacatcagtgatctggacgacctaaagcatcatcgggaatcgattattgagccccagttcgaccctttttgccatggccctgccccagccctttatatagacaaattctcgcgctttttcttcccgttttcgttctttatactcaatattgtctattggacaacgttcctatga

SEQ ID NO: 4 - protein, 422 aa, Drosophila melanogaster
MQSPTSKLVEFRCLIALAIYLHALEQSIQHCHCVHGYRNNTESAELVSHYESSLSLPDILPIPSKTYDKNRAPKLLGQPTVVYFHVTVLSLDSINEESMTYVTDIFLAQSWRDPRLRLPENMSEQYRILDVDWLHSIWRPDCFFKNAKKVTFHEMSIPNHYLWLYHDKTLLYMSKLTLVLSCAMKFESYPHDTQICSMMIESLSHTVEDLVFIWNMTDPLVVNTEIELPQLDISNNYTTDCTIEYSTGNFTCLAIVFNLRRRLGYHLFHTYIPSALIVVMSWISFWIKPEAIPARVTLGVTSLLTLATQNTQSQQSLPPVSYVKAIDVWMSSCSVFVFLSLMEFAVVNNFMGPVATKAMKGYSDENISDLDDLKHHRESIIEPQFDPFCHGPAPALYIDKFSRFFFPFSFFILNIVYWTTFL

SEQ ID NO: 5 - DNA, 1192 bp, Drosophila melanogaster
  CDS (1)..(1191)
atgcaaagcccaactagcaaattggtagaattcaggtgcctgattgcgttggcaatatatttgcacgcgctggagcaatcgatccagcactgccattgtgttcatggttacagaaataacacggagagcgccgagctggtctcccactacgagtcgagtctttcgctcccggacattttgcccattccctcaaagacgtacgacaagaaccgggctcccaagctcctcggccagcccacagtagtctacttccatgtcacggtcctctccctggactccattaacgaggagtctatgacctatgtgacggacatcttccttgcacaaagctggcgtgatcctcgcctgcggttgcctgagaacatgagtgagcagtatcgcatattggatgtcgactggttgcacagcatttggcggcccgattgcttctttaagaacgccaaaaaggtcaccttccatgagatgagcattcccaatcactatctctggctgtaccacgacaaaacgctgctctatatgtccaaactcacgttggtcctgtcgtgcgccatgaagtttgagtcctatccgcatgacacgcaaatctgctccatgatgatcgagagtttatcccatacggtggaagatttggttttcatttggaacatgaccgacccacttgtggttaacacggagattgagttgccgcagctagacatatcaaataactacacaaccgactgtactatagagtactcaacaggtaacttcacctgcctggccattgtgttcaacctgcgccgacgcctgggttaccatttgttccacacctacatcccctcggctctgattgtggtcatgtcttggatatcgttttggataaaaccagaagcgatacccgcccgtgtaactctgggagtgacctcactgctaaccctggccacccagaatacccagtcgcaacaatcgctgccgccggtttcgtatgtcaaggctatagacgtctggatgtcgtcctgttcggtgtttgtattcctttctctgatggaatttgcagtggtcaacaattttatgggaccggtggccacaaaggcaatgaaggggtattcggacgagaacatcagtgatctggacgacctaaagcatcatcgggaatcgattattgagccccagtacgacactttctgccatggccatgccacgccatttatat

SEQ ID NO: 6 - protein, 397 aa, Drosophila melanogaster
MQSPTSKLVEFRCLIALAIYLHALEQSIQHCHCVHGYRNNTESAELVSHYESSLSLPDILPIPSKTYDKNRAPKLLGQPTVVYFHVTVLSLDSINEESMTYVTDIFLAQSWRDPRLRLPENMSEQYRILDVDWLHSIWRPDCFFKNAKKVTFHEMSIPNHYLWLYHDKTLLYMSKLTLVLSCAMKFESYPHDTQICSMMIESLSHTVEDLVFIWNMTDPLVVNTEIELPQLDISNNYTTDCTIEYSTGNFTCLAIVFNLRRRLGYHLFHTYIPSALIVVMSWISFWIKPEAIPARVTLGVTSLLTLATQNTQSQQSLPPVSYVKAIDVWMSSCSVFVFLSLMEFAVVNNFMGPVATKAMKGYSDENISDLDDLKHHRESIIEPQYDTFCHGHATPFI

SEQ ID NO: 7 - DNA, 1272 bp, Drosophila melanogaster
  CDS (1)..(1269)
atggagtccgagtgcgtttgcccagtttcatcagatttacgccacagggctgttctgttccacatatgcagaaataacacggagagcgccgagctggtctcccactacgagtcgagtctttcgctcccggacattttgcccattccctcaaagacgtacgacaagaaccgggctcccaagctcctcggccagcccacagtagtctacttccatgtcacggtcctctccctggactccattaacgaggagtctatgacctatgtgacggacatcttccttgcacaaagctggcgtgatcctcgcctgcggttgcctgagaacatgagtgagcagtatcgcatattggatgtcgactggttgcacagcatttggcggcccgattgcttctttaagaacgccaaaaaggtcaccttccatgagatgagcattcccaatcactatctctggctgtaccacgacaaaacgctgctctatatgtccaaactcacgttggtcctgtcgtgcgccatgaagtttgagtcctatccgcatgacacgcaaatctgctccatgatgatcgagagtttatcccatacggtggaagatttggttttcatttggaacatgaccgacccacttgtggttaacacggagattgagttgccgcagctagacatatcaaataactacacaaccgactgtactatagagtactcaacaggtaacttcacctgcctggccattgtgttcaacctgcgccgacgcctgggttaccatttgttccacacctacatcccctcggctctgattgtggtcatgtcttggatatcgttttggataaaaccagaagcgatacccgcccgtgtaactctgggagtgacctcactgctaaccctggccacccagaatacccagtcgcaacaatcgctgccgccggtttcgtatgtcaaggctatagacgtctggatgtcgtcctgttcggtgtttgtattcctttctctgatggaatttgcagtggtcaacaattttatgggaccggtggccacaaaggcaatgaaggggtattcggacgagaacatcagtgatctggacgacctaaaggttgtgcggcagttacagattggacttcagccagttctgaaaaagaagttaagcaattacttcaaagttcctaaagccataataaatgtatttaccaatgatgcccttcggagcggtatcttcggaggtgtcatagtaacgaagttgatgcttaatagagtccaacgaaccatcgctgtttccagcccttga

SEQ ID NO: 8 - protein, 423 aa, Drosophila melanogaster
MESECVCPVSSDLRHRAVLFHICRNNTESAELVSHYESSLSLPDILPIPSKTYDKNRAPKLLGQPTVVYFHVTVLSLDSINEESMTYVTDIFLAQSWRDPRLRLPENMSEQYRILDVDWLHSIWRPDCFFKNAKKVTFHEMSIPNHYLWLYHDKTLLYMSKLTLVLSCAMKFESYPHDTQICSMMIESLSHTVEDLVFIWNMTDPLVVNTEIELPQLDISNNYTTDCTIEYSTGNFTCLAIVFNLRRRLGYHLFHTYIPSALIVVMSWISFWIKPEAIPARVTLGVTSLLTLATQNTQSQQSLPPVSYVKAIDVWMSSCSVFVFLSLMEFAVVNNFMGPVATKAMKGYSDENISDLDDLKVVRQLQIGLQPVLKKKLSNYFKVPKAIINVFTNDALRSGIFGGVIVTKLMLNRVQRTIAVSSP

SEQ ID NO: 9 - DNA, 1608 bp, Drosophila melanogaster
  CDS (1)..(1605)
atggtgttccaaataataatcctggtgatctgcaccatctgcatgaagcactacgccaaaggggagtttcaacaaagtctggccataaccgacatcctgcccgaggacatcaagcgttacgacaagatgagaccgccgaagaaagagggtcagccgacgatagtctacttccatgtgacggtgatgggtctggactccattgatgagaactcgatgacttatgtggcggatgtgttctttgcacagacgtggaaggatcatcgcctgcgattgccggagaatatgacacaggaataccgcctgctcgaggtggactggctaaaaaatatgtggcggccggattcgtttttcaaaaacgccaaatcggtgacctttcagaccatgacaatacccaatcactatatgtggctgtacaaggataagaccattctgtacatggtcaaactaacactgaagctgtcctgcatcatgaacttcgccatttatcctcatgatacacaggagtgcaagctgcaaatggaaagcctgtcccacaccacggatgacttgatattccagtgggatccaacaacgccccttgtggttgatgaaaacatcgaactgccgcaggtggccctcatccgaaatgaaacggcggactgtacccaagtttattccactggcaacttcacatgcctggaggtggtgttcacccttaagcgtcgtttggtttactacgttttcaacacctacattcccacctgcatgatagtgatcatgtcatgggtatccttctggatcaaaccggaggcggcaccagcccgtgtgactctgggtgtcacctccttgctaacgctttccacgcaacacgccaaatcgcagtcgtctttgccacctgtttcctatctcaaggcagtggacgcctttatgtccgtttgcacggtgttcgtgtttatggccctcatggagtattgtctaataaacatcgtcctgagcgacacgcccattcccaagccgatggcctatccacccaaacctgtggcgggagatgggcccaagaaagagggcgagggtgctcctcctgggggcagcaactcgacggccagcaagcaacaagccaccatgttgccactggccgatgaaaagatcgagaaaattgagaagatctttgacgagatgaccaagaatagaaggattgtaaccaccacacgccgcgtggtgcgtccgccattggacgccgatggtccgtggattccgcgacaggagtcgcggataatactgaccccgactatcgctccgccgccaccgccccctcagccagcggcaccggaaccggaactgcccaagccgaaactcacacccgcccaggagcggctcaagcgggctatatatatagatcggtcctcgcgcgtccttttccccgccctcttcgccagtctgaatggcatctactggtgtcaactttggatggaacaatggaacttgctcgattttctaccgcctgcagtcttcaaacagccgcagggtgaagagaaacagacttatgaatacccaattaattatgttggtcacacgctccacacttttgccttttcacggtcgttagtgacgctcgagcaaaaactatga

SEQ ID NO: 10 - protein, 535 aa, Drosophila melanogaster
MVFQIIILVICTICMKHYAKGEFQQSLAITDILPEDIKRYDKMRPPKKEGQPTIVYFHVTVMGLDSIDENSMTYVADVFFAQTWKDHRLRLPENMTQEYRLLEVDWLKNMWRPDSFFKNAKSVTFQTMTIPNHYMWLYKDKTILYMVKLTLKLSCIMNFAIYPHDTQECKLQMESLSHTTDDLIFQWDPTTPLVVDENIELPQVALIRNETADCTQVYSTGNFTCLEVVFTLKRRLVYYVFNTYIPTCMIVIMSWVSFWIKPEAAPARVTLGVTSLLTLSTQHAKSQSSLPPVSYLKAVDAFMSVCTVFVFMALMEYCLINIVLSDTPIPKPMAYPPKPVAGDGPKKEGEGAPPGGSNSTASKQQATMLPLADEKIEKIEKIFDEMTKNRRIVTTTRRVVRPPLDADGPWIPRQESRIILTPTIAPPPPPPQPAAPEPELPKPKLTPAQERLKRAIYIDRSSRVLFPALFASLNGIYWCQLWMEQWNLLDFLPPAVFKQPQGEEKQTYEYPINYVGHTLHTFAFSRSLVTLEQKL

SEQ ID NO: 11 - DNA, 1458 bp, Drosophila melanogaster
  CDS (1)..(1455)
atggtgttccaaataataatcctggtgatctgcaccatctgcatgaagcactacgccaaaggggagtttcaacaaagtctggccataaccgacatcctgcccgaggacatcaagcgttacgacaagatgagaccgccgaagaaagagggtcagccgacgatagtctacttccatgtgacggtgatgggtctggactccattgatgagaactcgatgacttatgtggcggatgtgttctttgcacagacgtggaaggatcatcgcctgcgattgccggagaatatgacacaggaataccgcctgctcgaggtggactggctaaaaaatatgtggcggccggattcgtttttcaaaaacgccaaatcggtgacctttcagaccatgacaatacccaatcactatatgtggctgtacaaggataagaccattctgtacatggtcaaactaacactgaagctgtcctgcatcatgaacttcgccatttatcctcatgatacacaggagtgcaagctgcaaatggaaagcctgtcccacaccacggatgacttgatattccagtgggatccaacaacgccccttgtggttgatgaaaacatcgaactgccgcaggtggccctcatccgaaatgaaacggcggactgtacccaagtttattccactggcaacttcacatgcctggaggtggtgttcacccttaagcgtcgtttggtttactacgttttcaacacctacattcccacctgcatgatagtgatcatgtcatgggtatccttctggatcaaaccggaggcggcaccagcccgtgtgactctgggtgtcacctccttgctaacgctttccacgcaacacgccaaatcgcagtcgtctttgccacctgtttcctatctcaaggcagtggacgcctttatgtccgtttgcacggtgttcgtgtttatggccctcatggagtattgtctaataaacatcgtcctgagcgacacgcccattcccaagccgatggcctatccacccaaacctgtggcgggagatgggcccaagaaagagggcgagggtgctcctcctgggggcagcaactcgacggccagcaagcaacaagccaccatgttgccactggccgatgaaaagatcgagaaaattgagaagatctttgacgagatgaccaagaatagaaggattgtaaccaccacacgccgcgtggtgcgtccgccattggacgccgatggtccgtggattccgcgacaggagtcgcggataatactgaccccgactatcgctccgccgccaccgccccctcagccagcggcaccggaaccggaactgcccaagccgaaactcacacccgcccaggagcggctcaagcgggctatatatatagatcggtcctcgcgcgtccttttccccgccctcttcgccagtctgaatggcatctactggtgtgtgttctacgagtatctataa

SEQ ID NO: 12 - protein, 485 aa, Drosophila melanogaster
MVFQIIILVICTICMKHYAKGEFQQSLAITDILPEDIKRYDKMRPPKKEGQPTIVYFHVTVMGLDSIDENSMTYVADVFFAQTWKDHRLRLPENMTQEYRLLEVDWLKNMWRPDSFFKNAKSVTFQTMTIPNHYMWLYKDKTILYMVKLTLKLSCIMNFAIYPHDTQECKLQMESLSHTTDDLIFQWDPTTPLVVDENIELPQVALIRNETADCTQVYSTGNFTCLEVVFTLKRRLVYYVFNTYIPTCMIVIMSWVSFWIKPEAAPARVTLGVTSLLTLSTQHAKSQSSLPPVSYLKAVDAFMSVCTVFVFMALMEYCLINIVLSDTPIPKPMAYPPKPVAGDGPKKEGEGAPPGGSNSTASKQQATMLPLADEKIEKIEKIFDEMTKNRRIVTTTRRVVRPPLDADGPWIPRQESRIILTPTIAPPPPPPQPAAPEPELPKPKLTPAQERLKRAIYIDRSSRVLFPALFASLNGIYWCVFYEYL

SEQ ID NO: 13 - DNA, 1491 bp, Drosophila melanogaster
  CDS (1)..(1488)
atggccaatggatcggtggtggtgtcgcctttgaacaccacggatgctttcagtgtgtccatcaacctgtcgcaatcgactgtgaacaattgtccttcgctgaaaaacgcagagtccatggcgctgatggagctgctgacgagactaaccgccccctgtcgctatgacaggatggttccacccgtggtccacaacaaagatggtgaggaggtacccatggacatatatgcccggttttacatatatgtgatgaagaacctggactcgagtgacctgcagtttacggtgcagggtctgctgcagttgcgatacctggacccgcgcctggccttctccagctatctgcctaaccgaaggcagcccataatgggggagtcggaactgaagaagatgctctgggtgccacatatattcctgaccaacgaacaggcctccactgtcctgggcaccagcgccaaggacgagctgaccagcatttatccgaatggcacagttcttacatccacgcggctgcaggccacactgtactgctggatgaacttccagaagtttccgtttgatgagcagaagtgtaagaccacattggaaagttggatgtacaacaccacgctggtgcaactccactgggagaccgataacccagtgagtttcgacaagcaacttcagctgacggaatacaacctaattgggtcgctgtacaacgaatccatccgagtgtccaacgaatcctacatgtcccatggctccttggagggcaactacagcatcattagcttcaccgttctgcttactcgggaagtgggctactacgtgatcgactactttctgccctcgatcatgatagtcaccatttcgtgggtgtccttctggctgcaggcggatcaaacccccgcaaggactactctgggctgcaccacgctcctatccttcatcactctgtccttgtcccaggagaacaatctgatgaaggtgagctacgtgaccatgtcggaggtgtggttcctggtctgcaccatcttcattttcggcagcctggtggagttcgcctttgtcaacaccatctggaggcgaaacaacgatctgcaactcaagaagcgaaccaccaagtatatagtcaagtccacatttgtcccacatctgaagaaacatcggcggcatggatatagaaggaccgatagcactatgagcaccatgagcaccaccagcatggacaagacctgcggtcccaacaacaccgtgatcaccatagagacaccaattattattggaggcagtcttagtcgcgaggactcggccattagcttggacgagcaggacgagacctccacctcggagtccagcgattcgtcgaaggagaaacccgcgcagaccttcgccaccatgacacccaaggaggtgtccctgtggatcgaccgcaagatgcggttcgtcttccctctgtccttcatcgtgttcaacgccttgttttggaccctggtctactgcctttga

SEQ ID NO: 14 - protein, 496 aa, Drosophila melanogaster
MANGSVVVSPLNTTDAFSVSINLSQSTVNNCPSLKNAESMALMELLTRLTAPCRYDRMVPPVVHNKDGEEVPMDIYARFYIYVMKNLDSSDLQFTVQGLLQLRYLDPRLAFSSYLPNRRQPIMGESELKKMLWVPHIFLTNEQASTVLGTSAKDELTSIYPNGTVLTSTRLQATLYCWMNFQKFPFDEQKCKTTLESWMYNTTLVQLHWETDNPVSFDKQLQLTEYNLIGSLYNESIRVSNESYMSHGSLEGNYSIISFTVLLTREVGYYVIDYFLPSIMIVTISWVSFWLQADQTPARTTLGCTTLLSFITLSLSQENNLMKVSYVTMSEVWFLVCTIFIFGSLVEFAFVNTIWRRNNDLQLKKRTTKYIVKSTFVPHLKKHRRHGYRRTDSTMSTMSTTSMDKTCGPNNTVITIETPIIIGGSLSREDSAISLDEQDETSTSESSDSSKEKPAQTFATMTPKEVSLWIDRKMRFVFPLSFIVFNALFWTLVYCL

SEQ ID NO: 15 - DNA, 1353 bp, Drosophila melanogaster
  CDS (1)..(1350)
atgtttctgttcgcagaatacgactctgatggaaaacgactgcccattgtggtgaagacgcgtatttatgtgtacttcctgcagaacctcaactccgacctgctgcagttcaagatgcacgcgctgttgcagctgcgatttcaggacaagcggctggcttacaaggccttcaatcgcagcgacaacatcctgggccagaagcacctgagcgaacgcctctggctgccgcacatcttctttgccaacgaacgagagtccagtatcctgggcacggacgagaaggacgtcctgacttcgctttcgcccgagggaaacgtgatcatctccacgcgcatgcaggccagtctctactgctggatgaactttcagaagtttccctttgaccagcagttctgctccacggtcctggagagctggatgtacaacacttccgatttgattctggagtgggagccgcacacgcccatctcctttgacccggaaatgaggctcactgagtacaatatggcccagttctggcacaacactacgattgtgcagtcggatggggacaatctgcggcatggagcttttgctggtaactatagttcgctgagctttacggtcaatctgaagcgagaaattggcttctatttgctcgactattacctgccctccatgatgatcgtggccatctcttgggtgtccttctggcttcaggcggacgcatctcctccaaggattatgctgggaaccagcaccatgttgtcgttcatcacgctttcctcatcgcagagcaagaacctgcccaaggtgagctacataaaggtgtcggaggtgtggtgcctgggctgcaccttcttcatcttcggcagcctggtggagtttgcctttgtcaacaccatttggcgccgcaaggagaacattgagctgaagaaggtcaacagcaagtacatcataaaatccacattgacaccacgaccggcccgccgtcaaattggcggaagtttgagcaacgagtccagggctagatcgtgttccagtttggacaacattgtttccagcacggagagcgtgcgcaatggcaatggcactgtgaatcagggcttcaacaactatttgacagtgcatcccaatttacccatcataagaacggagtgcgccgaggcagatacagtgtctatctgcagtgcgcgtacgaacaacgatcatatcgtggatgtggacaaggataaaaaggacacgccgcccacctttaccaccatgacaccgcaggagatcgccatgtggatagaccggcggtcgcgatttctttttccggccatgttcctcgccttcaacgctctgtattggaccttcgtctatgttttgtaa

SEQ ID NO: 16 - protein, 450 aa, Drosophila melanogaster
MFLFAEYDSDGKRLPIVVKTRIYVYFLQNLNSDLLQFKMHALLQLRFQDKRLAYKAFNRSDNILGQKHLSERLWLPHIFFANERESSILGTDEKDVLTSLSPEGNVIISTRMQASLYCWMNFQKFPFDQQFCSTVLESWMYNTSDLILEWEPHTPISFDPEMRLTEYNMAQFWHNTTIVQSDGDNLRHGAFAGNYSSLSFTVNLKREIGFYLLDYYLPSMMIVAISWVSFWLQADASPPRIMLGTSTMLSFITLSSSQSKNLPKVSYIKVSEVWCLGCTFFIFGSLVEFAFVNTIWRRKENIELKKVNSKYIIKSTLTPRPARRQIGGSLSNESRARSCSSLDNIVSSTESVRNGNGTVNQGFNNYLTVHPNLPIIRTECAEADTVSICSARTNNDHIVDVDKDKKDTPPTFTTMTPQEIAMWIDRRSRFLFPAMFLAFNALYWTFVYVL

SEQ ID NO: 17 - DNA, 1257 bp, Drosophila melanogaster
  CDS (1)..(1254)
atgctgagcgaagccacggaagcccacatgaactacacagccaagccgcgagtagttcttccgcctaattatgtcaaggaaatacgaccgccctcgaagaagggctcaccagtgatagtagactttagcatattcgttgtagatattaactcaattaacgtagaggatatggactttagagtagacatgtttatacatcagcgctggctggagtcgcgtctggagatcccagatgacatattcgaggagggcgacgactacgtgacgctgcttccagaggtctttgagaatttttggcagccggatccgtactttcttaattccaaaattgccgaaatagcgacactgacccacaagttcacgtcggtgacgctctacaagaacaaaacggtgcgctatgcggcccggatgcatgcgatcatcgcctgccagatggagttccagctgtaccccatggacatccaggtgtgtcccatctacatcgagagcttctcgtcgaacaatcaaaaggttaagctgcgctggtccgactccggggtgaccctcaatccggaactgaagctgctgcagtacaatctgggccagccgctggaactggaggagagcgacggctacatgccggagaaggtggggaacttctcccgcctcaccgtatacttccgcttcgagcgccagattggccaccacctcatccagaccttcgccccctcatcgctggtgcttctggctgggactggacgccattccggggcgcgtaacgctactggtgacctgcatgctgaccctggtgaccatgttacgggcgccgacattccgccagtggcctacgttaaggcactggatctctggatggccgggtgcatgctgtccgtgttcgccgcgctggccgagttcgtggtggtgaaagtgctggacgtgcagtaccagtaccaggtgaaccgcatacccaaggtactgcccatgcgcatcagcaatatggagaagggtcagtgtgcgacggtggccagttgggagggcggagcggtgcggtcgcggaaggccactcagacgcccacgacaccgggccagggtaacggcggaccgccgaagccagcaagacgtcaaagtctactttccgtggcgtggacggatacggataccggcgtggagaagatcatgtggcgagagatcgacaaggtgtcccgcgccgtattccccatcctgttctttgtgtttgtgctcctgtactggccgatactgctgatgaagtcgtcctag

SEQ ID NO: 18 - protein, 418 aa, Drosophila melanogaster
MLSEATEAHMNYTAKPRVVLPPNYVKEIRPPSKKGSPVIVDFSIFVVDINSINVEDMDFRVDMFIHQRWLESRLEIPDDIFEEGDDYVTLLPEVFENFWQPDPYFLNSKIAEIATLTHKFTSVTLYKNKTVRYAARMHAIIACQMEFQLYPMDIQVCPIYIESFSSNNQKVKLRWSDSGVTLNPELKLLQYNLGQPLELEESDGYMPEKVGNFSRLTVYFRFERQIGHHLIQTFAPSSLVLLAGTGRHSGARNATGDLHADPGDHVTGADIPPVAYVKALDLWMAGCMLSVFAALAEFVVVKVLDVQYQYQVNRIPKVLPMRISNMEKGQCATVASWEGGAVRSRKATQTPTTPGQGNGGPPKPARRQSLLSVAWTDTDTGVEKIMWREIDKVSRAVFPILFFVFVLLYWPILLMKSS

(19) United States
(12) Patent Application Publication
(10) Pub. No.: US 2002/0001824 A1
(43) Pub. Date: Jan. 3, 2002

(54) Ligand-gated anion channels of insects

(76) Inventors: Eva-Maria Franken, Leichlingen (DE); Gabi Friedrich, Leverkusen (DE); Klaus Raming, Redwood City, CA (US)

Correspondence Address: Patent Department, Bayer Corporation, 100 Bayer Road, Pittsburgh, PA, 15205-9741, US

(21) Appl. No.: 09/808,483

(22) Filed: Mar. 14, 2001

(30) Foreign Application Priority Data
Mar. 18, 2000 (DE) 10013619.2

(51) Int. Cl. A01K 67/033; C12N 9/00; C12N 5/06; C07H 21/04; C12P 21/02

(52) U.S. Cl. 435/69.1; 800/8; 435/348; 435/183; 536/23.2

(57) ABSTRACT

The invention relates to polypeptides which constitute new subunits of ligand-gated anion channels of insects, and to nucleic acids which encode these polypeptides and, in particular, to their use for finding active compounds for crop protection.

### FIELD OF THE INVENTION

The invention relates to ligand-gated anion channels of insects. More particularly, the invention relates to polypeptides which constitute novel subunits of ligand-gated anion channels of insects, to nucleic acids (also referred to as polynucleotides) encoding these polypeptides, and, in particular, to their use for finding active compounds for crop protection.

### BACKGROUND OF THE INVENTION

Ligand-gated anion channels, such as, for example, the chloride-dependent GABA receptor or the glutamate receptor, play an important role in neurotransmission in the animal kingdom. Binding of the ligand to the receptor causes temporary opening of the channel and allows the flux of anions through it. It is assumed that a receptor is composed of five subunits grouped around a pore. Each of these subunits is a protein composed of an extracellular N-terminal portion, followed by three transmembrane regions, an intracellular portion, and a fourth transmembrane region and a short extracellular C-terminal portion (Hosie et al., 1997).

In insects, GABA and glutamate are the most important inhibitory neurotransmitters of the central nervous system (Cleland, 1996; Delgado et al., 1989). Accordingly, GABA and glutamate receptors can be detected electrophysiologically on preparations of central ganglia of insects. These receptors are, moreover, the molecular target of important natural and synthetic insecticides (Sattelle, 1990, 1992; Cully et al., 1994, 1996; Arena et al., 1995), which act as agonists (for example avermectin) and antagonists (for example fipronil) of ligand-gated anion channels. In addition the absence of the chloride-dependent glutamate receptor in higher animals (vertebrates) emphasizes its outstanding importance as a selective target.

The protein sequence of a number of chloride-dependent GABA and glutamate receptors of insects is already known. Thus, for example, the sequences of three different GABA receptor subunits and of a glutamate receptor subunit have been described for Drosophila melanogaster (ffrench-Constant et al., 1991; Harvey et al., 1994; Henderson et al., 1993; Cully et al., 1996).

Providing new subunits of ligand-gated anion channels from insects is of great practical importance, for example, for the search for new insecticides, particularly interesting ligand-gated anion channels being those which differ to a greater extent from the known ones than is the case between functional homologues.

### SUMMARY OF THE INVENTION

Polypeptides of the present invention are involved in the synthesis of ligand-gated anion channels. Polynucleotides which encode the polypeptides may be used to form DNA constructs, vectors, host cells and transgenic invertebrates.

### DETAILED DESCRIPTION

The present invention is therefore based in particular on the object of providing subunits of ligand-gated anion channels of insects and assay systems based thereon with a high throughput of compounds to be assayed (High Throughput Screening Assays; HTS assays).

The object is achieved by providing polypeptides which form part of a ligand-gated anion channel and comprise an amino acid sequence which has at least 70% identity, preferably at least 80% identity, especially preferably at least 90% identity, very especially preferably at least 95% identity, with a sequence of SEQ ID NO: 2, 4, 6, 8, 10, 12, 14, 16 or 18 over a length of at least 20, preferably at least 25, especially preferably at least 30, consecutive amino acids and very especially preferably over their entire lengths.

The degree of identity of the amino acid sequences is preferably determined with the aid of the GAP program of the GCG package, Version 9.1, using standard settings (Devereux et al., 1984).

The term “polypeptides”, as used herein refers not only to short amino acid chains, which are usually termed peptides, oligopeptides or oligomers, but also to longer amino acid chains which are usually termed proteins. It encompasses amino acid chains which can be modified either by natural processes, such as post-translational processing, or by prior-art chemical methods. Such modifications may occur at different sites and repeatedly in a polypeptide, such as, for example, at the peptide backbone, at the amino acid side chain, at the amino and/or at the carboxyl terminus. They comprise, for example, acetylations, acylations, ADP-ribosylations, amidations, covalent linkages with flavins, haem moieties, nucleotides or nucleotide derivatives, lipids or lipid derivatives or phosphatidylinositol, cyclizations, disulphide bridge formations, demthylations, cystine formations, formylations, gamma-carboxylations, glycosylations, hydroxylations, iodinations, methylations, myristoylations, oxidations, proteolytic processings, phosphorylations, selenoylations and tRNA-mediated additions of amino acids.

The polypeptides according to the invention may exist in the form of “mature” proteins or as parts of larger proteins, for example as fusion proteins. They may furthermore exhibit secretion or leader sequences, pro-sequences, sequences which make possible simple purification, such as multiple histidine residues, or additional stabilizing amino acids.

A functional ligand-gated anion channel is preferably achieved by homo- or heteromerization of the polypeptides according to the invention.

The polypeptides according to the invention need not exhibit the length of naturally occurring anion channel subunits, but may also just be fragments thereof as long as they retain the ability of forming functional ligand-gated anion channels. Anion channels which, in comparison with channels composed of the polypeptides according to the invention with an amino acid sequence of SEQ ID NO: 2, 4, 6, 8, 10, 12, 14, 16 or 18, exert an activity which is increased or reduced by 50% are still considered as according to the invention. In this context, it is not necessary that the polypeptides according to the invention can be derived from ligand-gated anion channels of Drosophila melanogaster. Polypeptides which correspond to ligand-gated anion channels of, for example, the following invertebrates, or to fragments thereof which retain the ability of exerting the biological activity of these channels, are also considered as according to the invention: insects, nematodes, arthropods, molluscs.

In comparison with the corresponding region of naturally occurring ligand-gated anion channels, the polypeptides according to the invention may exhibit deletions or amino acid substitutions as long as they retain the ability of forming functional channels. Conservative substitutions are preferred. Such conservative substitutions encompass variations, one amino acid being replaced by another amino acid from amongst the following group:

1. Small aliphatic residues, unpolar residues or residues of little polarity: Ala, Ser, Thr, Pro and Gly;

2. Polar, negatively charged residues and amides: Asp, Asn, Glu and Gln;

3. Polar, positively charged residues: His, Arg and Lys;

4. Large aliphatic unpolar residues: Met, Leu, Ile, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| lle | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The term “functional ligand-gated anion channel” or “functional channel” as used herein refers to an anion channel which, after binding a ligand, becomes permeable to anions.

The subject-matter of the present invention further includes ligand-gated anion channels composed of the polypeptides according to the invention.

Preferred embodiments of the polypeptides according to the invention are those polypeptides which form part of ligand-gated anion channels of Drosophila melanogaster and which have an amino acid sequence of SEQ ID NO: 2, 4, 6, 8, 10, 12, 14, 16 or 18.

The present invention also relates to nucleic acid which encode the polypeptides according to the invention.

The nucleic acids according to the invention are, in particular, single-stranded or double-stranded deoxyribonucleic acids (DNA) or ribonucleic acids (RNA). Preferred embodiments are fragments of genomic DNA which may contain introns, and cDNAs.

cDNAs which have a nucleotide sequence of SEQ ID NO: 1, 3, 5, 7, 9, 11, 13, 15 or 17 constitute preferred embodiments of the nucleic acid according to the invention.

The present invention also encompasses nucleic acids which hybridize under stringent conditions with sequences of SEQ ID NO: 1, 3, 5, 7, 9, 11, 13, 15 or 17.

The term “to hybridize” as used in the present context describes the process during which a single-stranded nucleic acid molecule undergoes base pairing with a complementary strand. Starting from the sequence information disclosed herein, this allows, for example, DNA fragments to be isolated from insects other than Drosophila melanogaster which encode subunits of ligand-gated anion channels.

Preferred hybridization conditions are stated hereinbelow: Hybridization solution: 6X SSC/0% formamide, preferred hybridization solution: 6X SSC/25% formamide Hybridization temperature: 34° C., preferred hybridization temperature: 42° C. Wash step 1:2X SSC at 40° C., Wash step 2:2X SSC at 45° C.; preferred wash step 2. 0,6X SSC at 55° C.;

especially preferred wash step 2: 0.3X SSC at 65° C.

The present invention furthermore encompasses nucleic acids which have at least 70% identity, preferably at least 80% identity, especially preferably at least 90% identity, very especially preferably at least 95% identity, with a sequence of SEQ ID NO: 1, 3, 5, 7, 9, 11, 13, 15 or 17 over a length of at least 20, preferably at least 25, especially preferably at least 30, consecutive nucleotides, and very especially preferably over their full length.

The degree of identity of the nucleic acid sequences is preferably determined with the aid of the program GAP from the package GCG, Version 9.1, using standard settings.

The sequences in accordance with the Genbank Accession Nos. AC002502, AF145639 and AC004420 are incorporated into the present description by reference.

The present invention furthermore relates to DNA constructs which comprise a nucleic acid according to the invention and a heterologous promoter.

The term “heterologous promoter” as used in the present context refers to a promoter which has properties other than the promoter which controls the expression of the gene in question in the original organism. The term “promoter” as used in the present context generally refers to expression control sequences.

The choice of heterologous promoters depends on whether pro- or eukaryotic cells or cell-free systems are used for expression. Examples of heterologous promoters are the SV40 early or later promoter, the adenovirus early or late promoter or the cytomegalo virus early or late promoter, the lac system, the trp system, the main operator and promoter regions of phage lambda, the fd coat protein control regions, the 3-phosphoglycerate kinase promoter, the acid phosphatase promoter and the yeast α-mating factor promoter.

The invention furthermore relates to vectors which contain a nucleic acid according to the invention or a DNA construct according to the invention. All the plasmids, phasmids, cosmids, YACs or artificial chromosomes used in molecular biology laboratories can be used as vectors.

The present invention also relates to host cells containing a nucleic acid according to the invention, a DNA construct according to the invention or a vector according to the invention.

The term “host cell” as used in the present context refers to cells which do not naturally contain the nucleic acids according to the invention.

Suitable host cells are not only prokaryotic cells such as bacteria from the genera Bacillus, Pseudomonas, Streptomyces, Streptococcus, Staphylococcus, preferably E. coli, but also eukaryotic cells such as yeasts, mammalian cells, amphibian cells, insect cells or plant cells. Preferred eukaryotic host cells are HEK-293, Schneider S2, Spodoptera Sf9, Kc, CHO, COS1, COS7, HeLa, C127, 3T3 or BHK cells and, in particular, Xenopus oocytes.

The invention furthermore relates to antibodies which specifically bind to the abovementioned polypeptides or channels. Such antibodies are produced in the customary manner. For example, such antibodies may be produced by injecting a substantially immunocompetent host with such an amount of a polypeptide according to the invention or a fragment thereof which is effective for antibody production, and subsequently obtaining this antibody. Furthermore, an immortalized cell line which produces monoclonal antibodies may be produced in a manner known per se. If appropriate, the antibodies may be labelled with a detection reagent. Preferred examples of such a protection reagent are enzymes, radiolabelled elements, fluorescent chemicals or biotin. Instead of the complete antibody, fragments may also be employed which have the desired specific binding properties. The term “antibodies” as used in the present context therefore also extends to parts of complete antibodies, such as Fa, F(ab′)₂ or Fv fragments which are still capable of binding to the epitopes of the polypeptides according to the invention.

The nucleic acids according to the invention can be used, in particular, for generating transgenic invertebrates. These can be employed in assay systems which are based on an expression, of the polypeptides according to the invention, which deviates from the wild type. Based on the information disclosed herein, it is furthermore possible to generate transgenic invertebrates where expression of the polypeptides according to the invention is altered owing to the modification of other genes or promoters.

The transgenic invertebrates are generated, for example in the case of Drosophila melanogaster, by P-Element-mediated gene transfer (Hay et al., 1997) or, in Caenorhabditis elegans, by transposon-mediated gene transfer (for example by Tcl; Plasterk, 1996).

The invention thus also relates to transgenic invertebrates which contain at least one of the nucleic acids according to the invention, preferably transgenic invertebrates of the species Drosophila melanogaster or Caenorhabditis elegans, and to their transgenic progeny. The transgenic invertebrates preferably contain the polypeptides according to the invention in a form which deviates from the wild type.

The present invention furthermore relates to processes for producing the polypeptides according to the invention. Host cells which contain one of the nucleic acids according to the invention can be cultured under suitable conditions to produce the polypeptides encoded by the nucleic acids according to the invention. In doing this, the nucleic acid to be expressed may be adapted to the codon usage of the host cells. Thereupon, the desired polypeptides can be isolated from the cells or the culture medium in the customary manner. Polypeptides may also be produced in in vitro systems.

A rapid method of isolating the polypeptides according to the invention which are synthesized by host cells using a nucleic acid according to the invention starts with the expression of a fusion protein, it being possible for the fusion partner to be affinity-purified in a simple manner. For example, the fusion partner may be glutathione S-transferase. The fusion protein can then be purified on a glutathione affinity column. The fusion partner can then be removed by partial proteolytic cleavage, for example at linkers between the fusion partner and the polypeptide according to the invention to be purified. The linker can be designed such that it includes target amino acids such as arginine and lysine residues, which define sites for trypsin cleavage. To generate such linkers, standard cloning methods using oligonucleotides may be employed.

Other purification methods which are possible are based on preparative electrophoresis, FPLC, HPLC (for example using gel filtration, reversed-phase or moderately hydrophobic columns), gel filtration, differential precipitation, ion-exchange chromatography and affinity chromatography.

Since ligand-gated anion channels constitute membrane proteins, the purification methods preferably involve detergent extractions, for example using detergents which have no, or little, effect on the secondary and tertiary structures of the polypeptides, such as nonionic detergents.

The purification of the polypeptides according to the invention can encompass the isolation of membranes, starting from host cells which express the nucleic acids according to the invention. Such cells preferably express the polypeptides according to the invention in a sufficiently high copy number, so that the amount of polypeptide in a membrane fraction is at least 10 times higher than that found in comparable membranes of cells which naturally express ligand-gated anion channels; especially preferably, the amount is at least 100 times, very especially preferably at least 1000 times higher.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, especially preferably at least 100 times, higher than in a host cell preparation.

The polypeptides according to the invention may also be affinity-purified without a fusion partner with the aid of antibodies which bind to the polypeptides.

The present invention furthermore also relates to processes for the generation of the nucleic acids according to the invention. The nucleic acids according to the invention can be generated in the customary manner. For example, the complete nucleic acid molecules can be synthesized chemically. However, it is also possible to chemically synthesize only short sections of the sequences according to the invention, and such oligonucleotides can be radiolabelled or labelled with a fluorescent dye. The labelled oligonucleotides can be used for screening cDNA libraries generated starting from insect mRNA of for screening genomic libraries generated starting from insect genomic DNA. Clones which hybridize with the labelled oligonucleotides are chosen for isolating the DNA in question. After characterization of the DNA which has been isolated, the nucleic acids according to the invention are obtained in a simple manner.

Alternatively, the nucleic acids according to the invention can also be generated by means of PCR methods using chemically synthesized oligonucleotides.

The term “oligonucleotide(s)” as used in the present context denotes DNA molecules consisting of 10 to 50 nucleotides, preferably 15 to 30 nucleotides. They are synthesized chemically and can be used as probes.

The nucleic acids according to the invention allow new active compounds for crop protection and/or drugs for the treatment of humans and animals to be identified, such as chemicals which, being modulators, in particular agonists or antagonists, alter the properties of the ligand-gated anion channels according to the invention, to be identified. To this end, a recombinant DNA molecule comprising at least one nucleic acid according to the invention is introduced into a suitable host cell. The host cell is grown in the presence of a compound or a sample comprising a variety of compounds under conditions which allow expression of the receptors according to the invention. A change in the receptor properties can be detected as described hereinbelow in Example 2. This allows, for example, insecticidal substances to be found.

A functional ligand-gated anion channel can be detected by functionality assays or binding assays (Rudy et al., 1992). A functionality detection is, for example, the electrophysiological derivation of cells or oocytes which express ligand-gated anion channels and which respond to addition of ligands with an anion flux (which leads to changes in the potential at the cell membrane). Typically, the ligand-induced currents depend on the concentration of the ligand applied. Apart from the electrophysiological derivation, changes in the potential can also be visualized by, for example, potentiometric dyes. In a typical binding assay, potential agonists and antagonists are identified by their ability to displace known radiolabelled ligands from the channel. Examples of suitable known ligands are GABA, glutamate, glycine, muscimol, EBOB (n-propylethynyl bicycloorthobenzoate), BIDN (3,3-bis(trifluoromethyl)bicyclo-[2,2,1]heptane-2,2-dicarbonitrile), avermectin, fipronil.

Such assays can be used for finding agonists, antagonists or modulators in High Throughput Screening (HTS).

The term “agonist” as used in the present context refers to a molecule which activates ligand-gated anion channels.

The term “antagonist” as used in the present context refers to a molecule which displaces an agonist from its binding site.

The term “modulator” as used in the present invention constitutes the generic term for agonist and antagonist. Modulators can be small organochemical molecules, peptides or antibodies which bind to the polypeptides according to the invention. Further modulators may be small organochemical molecules, peptides or antibodies which bind to a molecule which, in turn, binds to the polypeptides according to the invention, thus influencing their biological activity. Modulators may constitute mimetics of natural substrates and ligands.

The modulators are preferably small organochemical compounds.

The binding of the modulators to the polypeptides according to the invention can alter the cellular processes in a manner which leads to the death of the insects treated therewith.

The present invention therefore also extends to the use of modulators of the polypeptides according to the invention or of the ligand-gated anion channels according to the invention as insecticides.

The nucleic acids according to the invention also allow compounds to be found which bind to the channels according to the invention. Again, they can be applied to plants as insecticides. For example, host cells which contain the nucleic acids according to the invention which express the corresponding channels or polypeptides, or the gene products themselves, are brought into contact with a compound or a mixture of compounds under conditions which permit the interaction of at least one compound with the host cells, the channels or the individual polypeptides.

Using host cells or transgenic invertebrates which contain the nucleic acids according to the invention, it is also possible to find substances which alter channel expression.

The above-described nucleic acids, vectors and regulatory regions according to the invention can furthermore be used for finding genes which encode polypeptides which are involved in the synthesis, in insects, of functionally similar ligand-gated anion channels. Functionally similar receptors are to be understood as meaning, in accordance with the present invention, channels which comprise polypeptides which, while differing from the amino acid sequence of the polypeptides described herein, essentially have the same functions.

Information on the sequence listing and on the figures:

SEQ ID NO: 1, 3, 5, 7, 9, 11, 13, 15 and 17 show the nucleotide and amino acid sequences of the isolated ligand-gated anion channel cDNAs. SEQ ID NO: 2, 4, 6, 8, 10, 12, 14, 16 and 18 furthermore show the amino acid sequences of the polypeptides deduced from the ligand-gated anion channel cDNA sequences.

### EXAMPLES

### Example 1

Isolation of the above-described polynucleotides

Polynucleotides were manipulated by standard methods of recombinant DNA technology (Sambrook et al., 1989). Nucleotide and protein sequences were bioinformatically processed using the package GCG Version 9.1 (GCG Genetics Computer Group, Inc., Madison Wis., U.S.A.).

### Example 2

Generation of the expression constructs

The sequence regions of SEQ ID NO: 1, 3, 5, 7, 9, 11, 13, 15 and 17 were amplified by means of polymerase chain reaction (PCR) and cloned into the vectors pcDNA3.1/Neo(lnvitrogen, Groningen) and plE1-¾ (Novagen, Madison Wis., U.S.A.).

Heterologous expression

The ligand-gated anion channels from insects were expressed functionally in Sf9 cell lines and Xenopus oocytes. To this end, expression vectors with the various subunits were introduced into the cells/oocytes individually and in various combinations.

Measurements in Sf9 cells

1. Cell culture

Sf9 cells were grown in 50 ml CELLSTAR cell culture flasks(Greiner, Nürtingen, Germany) at 26° C. in 5 ml of SF-900 II cell culture medium supplemented with L-glutamine (Gibco BRL, Karlsruhe, Germany).

2. Transfection

For transfection, 1×10⁵ cells were seeded per well of a 4-well NUNCLON cell culture dish (Nunc, Wiesbaden, Germany). Transfection was carried out with FuGENE 6 (Roche Molecular Biochemicals, Mannheim, Germany), following standard protocols. 1 μg of vector DNA was used per expression construct. The electrophysiological measurement was carried out 26 to 48 hours after transfection.

Oocyte measurements

1. Oocyte preparation the oocytes were obtained from an adult female Xenopus laevis from (Horst Khler, Hamburg, Germany). The frogs were kept in large tanks with circulating water at a water temperature of 20-24° C. Parts of the frog ovary were removed through a small incision in the abdomen (approx. 1 cm), with full anaesthesia. The ovary was then treated for approximately 140 minutes with 25 ml of collagenase (type l, , C-0130, SIGMA-ALDRICH CHEMIE GmbH, Deisenhofen, Germany; 355U/ml, prepared with Barth's solution without calcium in mM: NaCl 88, KCl 1, MgSO4 0.82, NaHCO3 2.4, Tris/HCl 5, pH7.4), with constant shaking. Thereupon, the oocytes were washed with Barth's solution without calcium. Only oocytes at maturity stage V (Dumont, 1972) were selected for the further treatment and transferred into microtitre plates (Nunc MicroWell™ plates, cat. No. 245128+263339 (lid), Nunc GmbH & Co. KG, Wiesbaden, Germany), filled with Barth's solution (in mM: NaCl 88, KCl 1, MgSO₄ 0.82, Ca(NO₃)₂ 0.33, CaCl₂ 0.41, NaHCO3 2.4, Tris/HCl 5, pH7.4) and gentamicin (gentamicin sulphate, G-3632, SIGMA-ALDRICH CHEMIE GmbH, Deisenhofen, Germany; 100U/ml). Thereupon, the oocytes were stored at 19.2° C. in a cooling incubator (type KB 53, WTB Binder Labortechnik GmbH, Tuttlingen, Germany).

2. Injecting the oocytes

Injection electrodes of diameter 10-15 μm, were prepared using a pipette-drawing device (type L/M-3P-A, List-electronic, Darmstadt-Eberstadt, Germany). Prior to injection, aliquots with the DNA were defrosted and diluted with water to a final concentration of 10 ng/μl. The DNA samples were centrifuged for 120 seconds at 3200 g (type Biofuge 13, Heraeus Instruments GmbH, Hanau, Germany). An extended PE tube was subsequently used as transfer tube to fill the pipettes from the rear end. The injection electrodes were attached to an X,Y,Z positioning system (treatment centre EP1090, isel-automation, Eiterfeld, Germany). With the aid of a Macintosh computer the oocytes in the microtitre plate wells were approached, and approximately 50 nl of the DNA solution were injected into the oocytes by briefly applying a pressure (0.5-3.0 bar, 3-6 seconds).

3. Electrophysiological measurements

A two-electrode voltage terminal equipped with a TURBO TEC-10CD (npi electronic GmbH, Tamm, Germany) amplifier was used to carry out the electrophysiological measurements. The micropipettes required for this purpose were drawn in two movements from aluminium silicate glass (capillary tube, Art.-No. 14 630 29, l=100 mm, Øₑₓₜ=1.60 mm, Øᵢₙₜ=1.22 mm, Hilgenberg GmbH, Malsfeld, Germany) (Hamill et al., 1981). Current and voltage electrodes had a diameter of 1-3 μm and were filled with 1.5M KCl and 1.5M potassium acetate. The pipettes had a capacitance of 0.2-0.5 MW. To carry out the electrophysiological measurements, the oocytes were transferred into a small chamber which was flushed continuously with normal Rimland solution (in mM: KCl 90, MgCl₂ 3, HEPES 5, pH 7.2). To apply a substance, the perfusion solution was exchanged for a substance solution with the same composition and additionally the desired substance concentration. To check the expression of the DNA, the holding potential (−60 mV) of the membrane was clamped in succession and stepped fashion onto 10 depolarizing and 10 hyperpolarizing potentials. The duration of the pulses was 100 ms. The voltage interval was 10 mV per step. Unresponsive oocytes were discarded. All the others were used for substance testing. The data were documented by means of a YT platter (YT platter, Model BD 111, Kipp & Zonen Delft BV, AM Delft, Netherlands). When test substances were assayed in concentration series, these measurements were carried out on at least two different oocytes and at least five different concentrations. The substances have been assayed directly and without preincubation for their antagonism. The individual data were entered in Origin (evaluation software, Microcal Software, Inc., Northampton, Mass. 01060-4410 U.S.A. (Additive GmbH, Friedrichsdorf/Ts, Germany). Means and standard deviations were calculated using Origin. These measurements were carried out at least in duplicate.

References:

Arena J. P. et al. (1995), The mechanism of action of avermectins in Caenorhabditis elegans: correlation between activation of glutamate-sensitive chloride current, membrane binding, and biological activity, J. Parasitol. 81 (2), 286-294

Cleland T. A. (1996), Inhibitory glutamate receptor channels, Mol. Neurobiol. 13 (2), 97-136

Cully D. F. et al. (1994), Cloning of an avermectin-sensitive glutamate-gated chloride channel from Caenorhabditis elegans, Nature 371, 707-711

Cully D. F. et al. (1996), Identification of a Drosophila melanogaster glutamate-gated chloride channel sensitive to the antiparasitic agent avermectin, J. Biol. Chem. 271 (33), 20187-20191

Delgado R. (1989), L-glutamate activates excitatory and inhibitory channels in Drosophila larval muscle, FEBS letters 243 (2), 337-342

Devereux et al. (1984), Nucleic Acids Research 12, 387

Dumont, J. N. (1972), Oogenesis in Xenopus laevis (Daudin). 1. Stages of oocyte development in laboratory maintained animals, J. Morphol. 136: 153-180

ffrench-Constant R. H. et al. (1991), Proc. Natl. Acad. Sci. U.S.A.88, 7209-7213

Harvey R. J. et al. (1994), Sequence of a Drosophila ligand-gated ion-channel polypeptide with an unusual amino-terminal extracellular domain, J. Neurochem. 62, 2480-2483

Hay et al. (1997), P element insertion-dependent gene activation in the Drosophila eye, Proceedings of The National Academy of Sciences of The United States of America 94 (10), 5195-5200

Henderson J. E. et al. (1993), Characterization of a putative gamma-aminobutyric acid (GABA) receptor beta subunit gene from Drosophila melanogaster, Biochem. Biophys. Res. Commun. 193, 474-482

Hosie A. M. et al. (1997), Molecular biology of insect neuronal GABA receptors, TINS 20, 578-583

Plasterk (1996), The Tcl/marinertransposon family, Transposable Elements/Current Topics in Microbiology and Immunology 204,125-143

Rudy et al., eds. (1992), Methods in Enzymology 207, Academic Press, Inc., San Diego, Calif.

Sambrook et al. (1989), Molecular Cloning, A Laboratory Manual, 2nd ed. Cold Spring Harbour Press

Sattelle D. B. (1990), GABA Receptors of Insects, Advances in Insect Physiology 22, 1-113

Satelle D. B. (1992), Receptors for L-glutamate and GABA in the nervous system of an insect (Periplaneta americana), Comp. Biochem. Physiol. C. 103 (3), 429-438

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. An isolated polypeptide which is involved in the synthesis of a ligand-gated anion channel and which comprises an amino acid sequence which has at least 70% identity with a sequence selected from SEQ ID NOS: 2, 4, 6, 8, 10, 12, 14, 16 and 18.

2. An isolated polypeptide according to claim 1, wherein the polypeptide has an amino acid sequence which corresponds to a sequence selected from SEQ ID NO: 2, 4, 6, 8, 10, 12, 14, 16 and 18.

3. A ligand-gated anion channel comprising a polypeptide according to claim 1.

4. An isolated polynucleotide comprising a nucleotide sequence which encodes a polypeptide according to claim 1.

5. An isolated polynucleotide according to claim 4, wherein the polynucleotide is a single-stranded or double-stranded DNA or RNA.

6. An isolated polynucleotide according to claim 5, wherein the polynucleotide is a fragment of genomic DNA or cDNA.

7. An isolated polynucleotide according to claim 4, wherein the polynucleotide has a nucleotide sequence which corresponds to a sequence selected from SEQ ID NO: 1, 3, 5, 7, 9, 11, 13, 15 and 17.

8. An isolated polynucleotide according to claim 4, wherein the polynucleotide hybridizes under stringent conditions with a sequence selected from SEQ ID NO: 1, 3, 5, 7, 9, 11, 13, 15 and 17.

9. A DNA construct comprising an isolated polynucleotide according to claim 4 and a heterologous promoter.

10. A vector comprising an isolated polynucleotide according to claim 4.

11. A vector according to claim 10, wherein the polynucleotide is linked functionally to one or more regulatory sequences which ensure the expression of the polynucleotide in pro- or eukaryotic cells.

12. A host cell comprising an isolated polynucleotide according to claim 4.

13. A host cell according to claim 12, wherein the host cell is a prokaryotic cell.

14. A host cell according to claim 12, wherein the host cell is an eukaryotic cell.

15. An antibody which binds specifically to a polypeptide according to claim 1.

16. A transgenic invertebrate containing an isolated polynucleotide according to claim 4.

17. A transgenic invertebrate according to claim 16, wherein the transgenic invertebrate is Drosophila melanogaster or Caenorhabditis elegans.

18. A transgenic progeny of an invertebrate according to claim 16.

19. A method of producing an isolated polypeptide according to claim 1, comprising the steps of:
(a) culturing a host cell comprising a polynucleotide comprising a nucleotide sequence which encodes a polypeptide according to claim 1 under conditions which ensure the expression of the polynucleotide, and
(b) obtaining the polypeptide from the cell or the culture medium.

20. A method of generating an isolated polynucleotide according to claim 4, selected from the group consisting of:
(a) completely chemically synthesizing the polynucleotide,
(b) chemically synthesizing oligonucleotides, labelling the oligonucleotides, hybridizing the oligonucleotides with DNA of a genomic library or cDNA library generated from insect genomic DNA or insect mRNA, respectively, selecting positive clones and isolating the hybridizing DNA from positive clones, and
(c) chemically synthesizing oligonucleotides and amplifying target DNA by PCR.

21. A method of generating a transgenic invertebrate, which comprises introducing an isolated polynucleotide according to claim 4 into an invertebrate.

22. A method of finding active compounds which alter the properties of a polypeptide according to claim 1, comprising the steps of:
(a) providing a host cell comprising a polynucleotide comprising a nucleotide sequence which encodes a polypeptide according to claim 1,
(b) culturing the host cell in the presence of a chemical or of a mixture of chemicals, and
(c) detecting altered properties of the polypeptide.

23. Method of finding a chemical which binds to a polypeptide according to claim 1, comprising the steps of:
(a) contacting a polypeptide according to claim I with a chemical or a mixture of chemicals under conditions which permit the interaction of a chemical with the polypeptide, and
(b) determining the chemical which binds specifically to the polypeptide.

24. A method of finding a chemical which alters the expression of a polypeptide according to claim 1, comprising the steps of:
(a) contacting a host cell comprising a nucleic acid comprising a nucleotide sequence which encodes a polypeptide according to claim 1 with a chemical or a mixture of chemicals,
(b) determining the concentration of the polypeptide according to claim 1, and
(c) determining the chemical which specifically influences the expression of the polypeptide.

25. A method of killing insects comprising the step of applying a modulator of a polypeptide according to claim 1 to insects and/or their habitats.

26. A method of killing insects comprising the step of applying a modulator of a ligand-gated anion channel according to claim 3 to insects and/or their habitats.

27. A vector comprising a DNA construct according to claim 9.

28. A host cell comprising a DNA construct according to claim 9.

29. A host cell comprising a vector according to claim 10.

30. An antibody which binds specifically to ligand-gated anion channel according to claim 3.

31. A method of producing a polypeptide according to claim 1, comprising the steps of:
(a) culturing a host cell comprising a DNA construct comprising a polynucleotide comprising a nucleotide sequence which encodes a polypeptide according to claim 1 and a heterologous promoter under conditions which ensure the expression of the polynucleotide, and
(b) obtaining the polypeptide from the cell or the culture medium.

32. A method of producing a polypeptide according to claim 1, comprising the steps of:
(a) culturing a host comprising a vector comprising a polynucleotide comprising a nucleotide sequence which encodes a polypeptide according to claim 1 under conditions which ensure the expression of the polynucleotide, and
(b) obtaining the polypeptide from the cell or the culture medium.

33. A method of producing a polypeptide according to claim 1, comprising the steps of:
(a) culturing a host comprising a vector a vector comprising a DNA construct comprising (i) an isolated polynucleotide comprising a nucleotide sequence which encodes a polypeptide according to claim 1 and (ii) a heterologous promoter, under conditions which ensure the expression of the polynucleotide, and
(b) obtaining the polypeptide from the cell or the culture medium.

34. A method of producing a polypeptide according to claim 1, comprising the steps of:
(a) expressing a polynucleotide comprising a nucleotide sequence which encodes a polypeptide according to claim 1 in an in vitro system, and
(c) obtaining the polypeptide from the in vitro system

35. A method of generating a transgenic invertebrate, which comprises introducing a vector according to claim 10 into an invertebrate

36. A method of finding active compounds which alter the properties of a polypeptide according to claim 1, comprising the steps of:
(a) providing a host cell comprising a DNA construct comprising (i) a polynucleotide comprising a nucleotide sequence which encodes a polypeptide according to claim I and (ii) a heterologous promoter,
(b) culturing the host cell in the presence of a chemical or of a mixture of chemicals, and
(c) detecting altered properties the polypeptide.

37. A method of finding active compounds which alter the properties of a polypeptide according to claim 1, comprising the steps of:
(a) providing a host cell comprising a vector, wherein the vector comprises a polynucleotide comprising a nucleotide sequence which encodes a polypeptide according to claim 1,
(b) culturing the host cell in the presence of a chemical or of a mixture of chemicals, and
(c) detecting altered properties of the polypeptide.

38. A method of finding a chemical which binds to a ligand-gated anion channel according to claim 3, comprising the steps of:
(a) contacting a ligand-gated anion channel according to claim 3 with a chemical or a mixture of chemicals under conditions which permit the interaction of a chemical with the polypeptide, and
(b) determining the chemical which binds specifically to the polypeptide.

39. A method of finding a chemical which alters the expression of a polypeptide according to claim 1, comprising the steps of:
(a) contacting a transgenic invertebrate containing a polynucleotide comprising a nucleotide sequence which encodes a polypeptide according to claim 1 with a chemical or a mixture of chemicals,
(b) determining the concentration of the polypeptide according to claim 1, and
(c) determining the chemical which specifically influences the expression of the polypeptide.
